(19) United States
(12) Patent Application Publication
(10) Pub. No.: US 2014/0154796 A1
(43) Pub. Date: Jun. 5, 2014

(54) Sampling Device and Method for Collection and Preservation of Live Cells from Tissues and Cell Cultures

(71) Applicants: Stanislav L. Karsten, Signal Hill, CA (US); Lili C. Kudo, Signal, CA (US)

(72) Inventors: Stanislav L. Karsten, Signal Hill, CA (US); Lili C. Kudo, Signal, CA (US)

(21) Appl. No.: 14/095,661

(22) Filed: Dec. 3, 2013

Related U.S. Application Data
(60) Provisional application No. 61/732,711, filed on Dec. 3, 2012.

(51) Int. Cl. C12Q 1/24

(52) U.S. Cl. CPC C12Q 1/24
USPC 435/304.1; 435/309.1

(57) ABSTRACT

A sampling apparatus for collection and preservation of live cells comprising a collection tube, a capillary tube extending distally and substantially axially from the collection tube, the capillary tube having a first capillary tube section defining a first capillary tube inside diameter, and a luer hub engaged proximally and substantially axially on the collection tube substantially opposite the capillary tube, the luer hub having a luer passage formed therethrough, whereby installation of the sampling apparatus in a linear actuator assembly capable of providing a vacuum as by engaging the luer hub with a filter coupling of the linear actuator assembly enables selective suctioning through the luer passage, the collection tube, and the capillary tube so as to collect one or more cells within the collection tube of the sampling apparatus.

### RELATED APPLICATIONS

This application claims priority and is entitled to the filing date of U.S. Provisional application Ser. No. 61/732,711, filed on Dec. 3, 2012 and entitled “Sampling device and method for collection and preservation of live cells from tissues and cell cultures.” The contents of the aforementioned application are incorporated by reference herein.

### BACKGROUND OF THE INVENTION

1. Incorporation by Reference

Applicant(s) hereby incorporate herein by reference any and all patents and published patent applications cited or referred to in this application.

2. Field of the Invention

Aspects of this invention relate generally to sampling devices and methods, and more particularly to such a device and method for collection and preservation of live cells from tissues and cell cultures.

3. Description of Related Art

The field of the invention is cell and cell cluster collection; more specifically, the present invention relates to direct collection of specific live cells from heterogeneous tissues and cell cultures for their further analysis (e.g. immunocytochemistry or isolation of macromolecules such as DNA, RNA or proteins) and sub-cultivation (e.g. primary cultures). Composition of methods and device developed for the collection of the specific exemplary live cells presented in this non-provisional patent application can be used in conjunction with the capillary-based cell and tissue acquisition system (“CTAS”) as disclosed in WO/2008/021202, which is incorporated herein by reference in its entirety. Combination of the presented device and methods for the collection of live cells with CTAS results in a generation of a CTAS-Live concept.

All referenced patents, applications and literature are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. The invention may seek to satisfy one or more of the above-mentioned desires. Although the present invention may obviate one or more of the above-mentioned desires, it should be understood that some aspects of the invention might not necessarily obviate them.

The following summary describes aspects of the present state of this field:

Cell specific analysis represents one of the leading technologies in biological science and is critical to the sound elucidation of cellular functions, which cannot be acquired from measurements at bulk population level. This is especially important for functional studies of the specific cell types that demand their accurate acquisition and sub-cultivation. Primary cell culture is a powerful tool to study cellular functions in vitro, providing valuable insights into their functions in vivo. However, collecting live single cells from cultures or tissues has remained challenging. It is especially challenging when live cells have to be collected from heterogeneous tissue sources for the purpose of their further sub-culturing as primary cultures. This is of particular importance in the field of stem cell research where numerous lines of progenitor cells residing in specific anatomical areas with a specific program to commit into particular cell types.

To summarize there are at least three main areas of application where specific live cells have to be collected. First, it is acquisition of live cells or cell clusters from tissues for the purpose of their re-culturing (e.g. stem cells). Second, collection of the specific live cells from cell cultures for the purpose of their re-culturing (e.g. clonal analysis) is often desired. And finally, acquisition of live cells and cell clusters from either tissues or cell cultures for the purpose of the analysis of cellular biological content is a further context. In this respect, collection of live cells ensures minimal damage and preserves specific profiles of the macromolecules to be analyzed. In recent years, single cell analysis has appeared as a novel frontier in life and biomedical sciences, particularly for omics studies (Fritzsch et al, 2012; Kalisky et al, 2011; Wang and Bodovitz, 2010; Wu and Singh, 2012). Single cell analysis reduces biological noise from the heterogeneous background, providing fundamental improvements for elucidating cellular diversity and heterogeneity. It is particularly useful in stem cell biology (Gobaa et al, 2011; Hope and Bhatia, 2011; Kobel and Lutolf, 2010), where understanding the functional properties of pluripotent or committed progenitors and differentiated cells within normally heterogeneous population requires the isolation of single cells for subculture and clonal expansion.

Cell specific analysis plays a central role in clinical diagnostics, drug discovery, molecular studies and the practice of medicine. Because most of the diseases affect specific cell types, selective analysis of individual cells, groups of cells or subanatomical parts from normally heterogeneous tissues is a prerequisite for sound molecular studies. The mixtures of different cell types result in “averaging out” of results, masking disease-specific changes pronounced only in specific subanatomical regions or cell types. This is a particularly important issue in neuroscience where brain tissues demonstrate incredible complexity and a disease usually affects only specific brain regions, cells or cell types, posing a remarkable challenge for understanding basic brain functions or the drug discovery process. Moreover, specific progenitors also reside only in specific areas; therefore, methods permitting their accurate acquisition are prerequisite. While several technologies have been developed permitting the acquisition of specific cells from already dead tissues, the technologies allowing obtaining specific live cells are very limited. This is particularly challenging when complex heterogeneous tissues have to be used.

There are several techniques ranging from manual microdissection to laser assisted technologies, such as micropunching and laser capture microdissection, which can be adopted for the collection of live cells. In addition, a capillary-based vacuum-assisted cell and tissue acquisition system (CTAS-Live) was recently developed based on WO/2008/021202. Manual tissue dissection may be performed on live tissues placed on non-coated glass slides. It is gross technology that is time-consuming, operator dependent and has a high risk of contamination. However, it can be applied at the cellular resolution. Laser-assisted microdissection techniques cope with high tissue complexity, reduce risks of contamination and increase reproducibility of cell procurement. However, its use with live cells and tissues is limited as it usually requires tissue pretreatment procedure. Therefore, it cannot be used with native (live) tissues effectively. Fluorescence assisted cell sorting instruments are capable of separating a heterogeneous suspension of cells into purified fractions on the basis of fluorescence and light scattering properties. Cells with the specific fluorescent signals can be directed in the collection tube and used for further analysis. Live cells may be collected if genetic GFP marker is expressed. However, one of the drawbacks of this technology is the invasive nature of tissue dissociation resulting in cell death and inability to separate specific live cells from adult heterogeneous tissues. In addition, both laser-assisted microdissection instruments and flow sorting machines are usually very expensive, limiting accessibility to these technologies for many research groups. In conclusion, most of the existing methods are unable to precisely collect live single cells in situ from native tissues or cell cultures.

Aspects of the present invention fulfill these needs and provide further related advantages as described in the following summary.

### SUMMARY OF THE INVENTION

Aspects of the present invention teach certain benefits in construction and use which give rise to the exemplary advantages described below.

More particularly, aspects of the current invention relate to a collection/sampling cartridge designed for the use in conjunction with a capillary-based cell and tissue acquisition system (CTAS) as disclosed in WO/2008/021202. The sampling cartridge consists of the glass capillary, collection tube with the cell culture medium or any buffer that ensures cell viability during the dissection procedure, and a part permitting attachment of the device to the CTAS linear actuator head (e.g. Luer hub; FIGS. 1 and 2). The device may be generally described as a disposable capillary unit for the collection of live cells or “DCU-Live.”

Several representative versions of DCU-Live designed to handle various volumes of the collected cells are presented in FIG. 1. Their main principle is based on the collection of the desired live cell or cell clusters (FIG. 3) via capillary with a predetermined diameter that permits collection of the desired tissue areas or cells and groups of cells from the cell cultures. The cells vacuumed in by the CTAS travel through the capillary barrel and end up in the collection tube with the cell culture medium (FIG. 1). Collected cells may remain in the collection chamber for the time of dissection or longer and then transferred to cell culture plates or any other vessel where cells may remain alive or grow until further experimentation or storage. DCU-Live in combination with CTAS permits isolation of live cells from heterogeneous tissue sources (e.g. brain) or cell cultures without affecting cell viability (FIG. 3) or cellular content.

A primary object of the present invention is to provide means of collecting specific live cells from complex native tissues and cell cultures for the purpose of their re-cultivation or extraction of cellular material. Live cell specific collection may be performed with DCU-Live via capillary-based vacuum-assisted cell and tissue acquisition system (“CTAS”) operated at the settings sufficient for the collection of live cells ensuring their overall viability and integrity of intracellular material as disclosed in WO/2008/021202.

Another object of the present invention is to provide a device that substantially precisely collects live single cells in situ from native tissues or cell cultures that is relatively inexpensive and relatively less invasive than pre-existing collection devices.

Another object of the present invention is to decrease the invasive nature of tissue dissociation resulting in cell death and inability to separate specific live cells from adult heterogeneous tissues.

Alternatively, the described exemplary “DCU-Live” for collecting live cells may utilize other methods for collecting live cells such as a continuous vacuum or any vacuum source not related with the cell and tissue acquisition system, as well as any volume displacement- or plunger-based devices. For example, DCU-Live may be connected to a tube connected to the vacuum source and collection may be performed under direct microscopic visualization with DCU-Live held by a micromanipulator or any other device permitting relatively precise positioning of the DCU-Live above the area of interest and permitting its positioning in the area of interest (cells).

In this respect, before explaining at least one embodiment of the invention in detail, it is to be understood that the invention is not limited in its application to the details of the construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting. Relative to the accomplishment of one or more of the above and related objects, this invention may be embodied in the form illustrated in the accompanying drawings, attention being called to the fact, however, that the drawings are illustrative only, and that changes may be made in the specific construction illustrated. It should be noted that the drawing figures may be in simplified form and might not be to precise scale.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1A is a side schematic view of an exemplary sampling apparatus, in accordance with at least one embodiment;

FIG. 1B is a side view thereof;

FIG. 1C is a side view of an alternative embodiment thereof;

FIG. 1D is a side view of a further alternative embodiment thereof;

FIG. 2 is a perspective view in reduced scale of the exemplary sampling apparatus of FIGS. 1A and 1B operably engaged with an exemplary linear actuator assembly;

FIGS. 3A and 3B are schematic views of first exemplary cell cultures before and after collection, respectively;

FIGS. 4A and 4B are schematic views of second exemplary cell cultures before and after collection, respectively; and

FIGS. 5A-5D are schematic views of third exemplary cell cultures after collection and re-cultivation.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION OF THE INVENTION

The above described drawing figures illustrate aspects of the invention in at least one of its exemplary embodiments, which are further defined in detail in the following description.

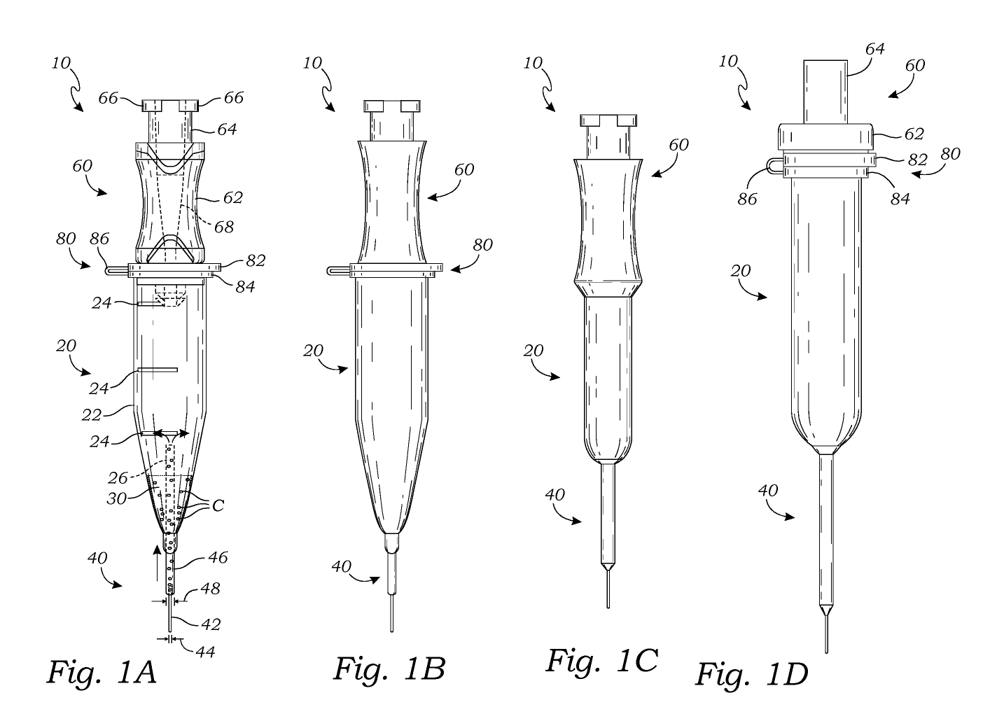

Turning now to FIG. 1A, there is shown a side schematic view of an exemplary embodiment of a sampling apparatus 10 configured as a disposable capillary unit for acquisition of live cells—hence, “DCU-Live.” The apparatus 10 comprises, in one embodiment, a collection tube 20, a capillary tube 40 extending substantially axially from the distal end thereof, and a luer hub 60 installed substantially axially on the proximal end of the collection tube 20. An optional folding cap 80 is installed between the collection tube 20 and the luer hub 60, as shown in the embodiment of FIGS. 1A and 1B and of FIG. 1D, corresponding to “medium” and “large” DCU-Live sampling devices but not in FIG. 1C corresponding to a “small” sampling device. The folding cap 80 may also be replaced with other such connectors now or later known and used in the art, such as a screw cap (not shown), for example. More generally, those skilled in the art will appreciate that all such components and their particular geometries, materials, and means of engagement may vary and may employ any suitable constructions now known or later developed in the art without departing from the spirit and scope of the invention.

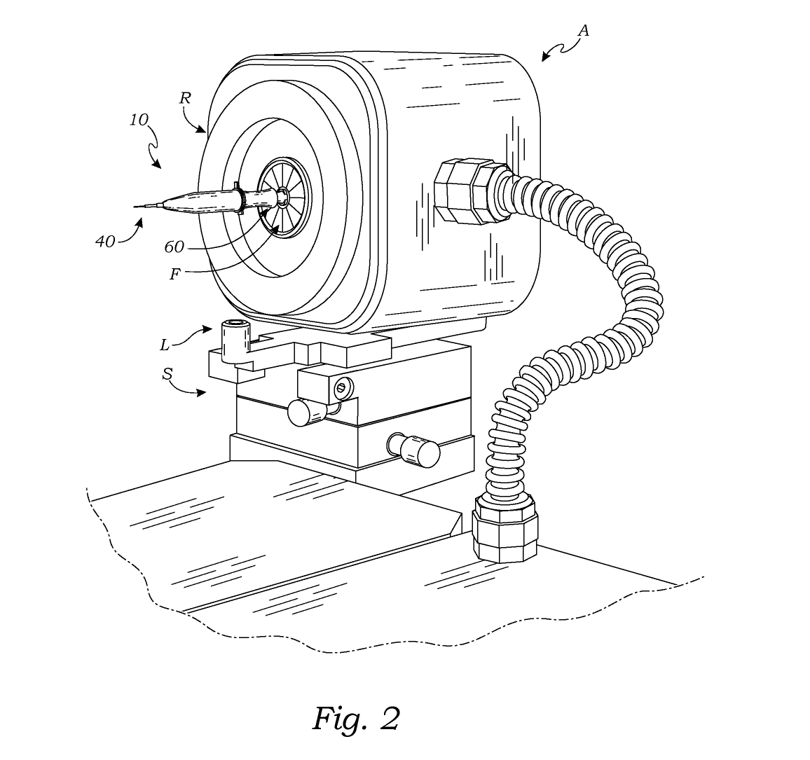

In a bit more detail, and with continued reference to FIG. 1A, the illustrated DCU-Live sampling apparatus 10 is shown as again having the capillary tube 40 substantially axially affixed within the distal end of the collection tube 20. Particularly, the capillary tube 40 is formed having a relatively smaller first capillary tube section 42 having a first capillary tube inside diameter 44 stepping up to a relatively larger second capillary tube section 46 having a second capillary tube inside diameter 48. As shown, the second capillary tube section 46 is received within the collection tube 20 so as to transition to or form a collection tube buffer 26, or tube portion protruding into or within the collection tube 20. It will be appreciated that the capillary tube 40, and the second capillary tube section 46, specifically, may be formed integrally or contiguous with the collection tube buffer 26 or the two may be separately formed and installed components, such as might be the case if the collection tube 20 is formed of molded plastic and so might have the collection tube buffer 26 formed integrally therewith, while the capillary tube 40 may be formed of glass. Even so, the second capillary tube section 46 may be formed of sufficient length so as to be inserted and extend within the collection tube 20 such that, again, the capillary tube 40 may be integral with the collection tube buffer 26, such that the assembly is formed all of glass in the exemplary embodiment. In either case, to install the capillary tube 40 in the collection tube 20, an adhesive or sealant (not shown) of any suitable kind now known or later developed in the art may be employed. Once the apparatus 10 is so configured, within the collection tube 20 there is deposited substantially at the distal end thereof about the collection tube buffer 26 a growth medium 30 in which any collected cells C may survive until further work with the cells C is to be performed. As also shown, the collection tube 20 may be formed having graduations or markings 24 on the collection tube body 22 for a quick visual determination of the volume of media or cells or cell culture materials that have been acquired, more about which is said below in connection both with both the alternative exemplary embodiments of the apparatus 10 and its use. Regarding the proximal or “coupling” end of the apparatus 10, again, there is installed a luer hub 60 in substantial axial alignment with the collection tube 20 and the capillary tube 40 for the purpose of removably engaging the sampling apparatus 10 with a sample acquisition system such as the live cell and tissue acquisition system (“CTAS”) as disclosed in WO/2008/021202 (“CTAS-Live”). Accordingly, the luer hub 60 is shown as being formed having a relatively larger first luer hub boss 62 configured for engagement with the open end or mouth of the collection tube 20, again, whether directly or via a folding cap 80 or the like, and from which first luer hub boss 62 extends substantially axially and proximally a relatively smaller second luer hub boss 64 that is itself formed with luer hub engagement tabs 66 for keyed engagement with any receptacle in which the apparatus 10 is to be removably installed, such as the filter coupling F of the CTAS-Live linear actuator assembly A as shown in FIG. 2. The luer hub 60 is further formed having a luer hub passage 68 that communicates substantially axially between opposite ends of the luer hub 60, or is a through-hole running the length of the luer hub 60 between or through the first and second luer hub bosses 62, 64. It will be appreciated that such a luer hub passage 68 enables airflow therethrough, such as in the case of a vacuum source within the CTAS-Live linear actuator assembly A employed to provide suction and thereby collection of cells in the collection tube 20 as again described further below. Once more, the luer hub 60 may be installed directly on or even be formed integrally with the collection tube 20 (see FIG. 1C) or may be engaged with the collection tube 20 via the folding cap 80 or some other such cap or coupling device now known or later developed. In the case of the folding cap 80 shown, there is an upper first folding cap member 82 that is engaged with the luer hub 60, and particularly the first luer hub boss 62, and a lower second folding cap member 84 that is engaged with the collection tube 20, and particularly the collection tube body 22. The first and second folding cap members 82, 84 are shown as being connected along one edge by a folding cap hinge 86, which it will be appreciated facilitates removal of the luer hub 60 and first folding cap member 82 from the collection tube 20 and second folding cap member 84 with the luer hub 60 and first folding cap member 82 then being pivoted away from the opening of the collection tube 20 so as to provide access thereto, such as to insert the growth medium 30 if not already packaged or placed within the collection tube 20 or to access and remove collected cells C and related material. In the exemplary embodiment shown in FIGS. 1A and 1B, a nominal “medium” size sampling apparatus 10 is shown, which in the exemplary embodiment has a chamber volume of approximately 0.2 ml. By comparison, and turning briefly to FIG. 1C, there is shown a nominal “small” size sampling apparatus 10 that in the exemplary embodiment has a chamber volume of approximately 50 μl. And in FIG. 1D, there is shown a nominal “large” size sampling apparatus 10 that in the exemplary embodiment has a chamber volume of approximately 0.5 ml. As shown in FIG. 1D, the luer hub 60, and particularly the second luer hub boss 64 may not be formed with engagement tabs 66 (FIG. 1A), but may instead have a substantially smooth wall, whether or not tapered, for an interference fit or engagement with a coupling device of the liner actuator assembly A such as the filter F. Again, those skilled in the art will appreciate that all such sizes or volumes are merely illustrative of features and aspects of the present invention and are expressly non-limiting. Relatedly, and by comparison, in the exemplary embodiment across all sizes of sampling apparatuses 10 shown, the DCU-Live internal diameter (“ID”), or the first capillary tube inside diameter 44, is a nominal 15 μm.

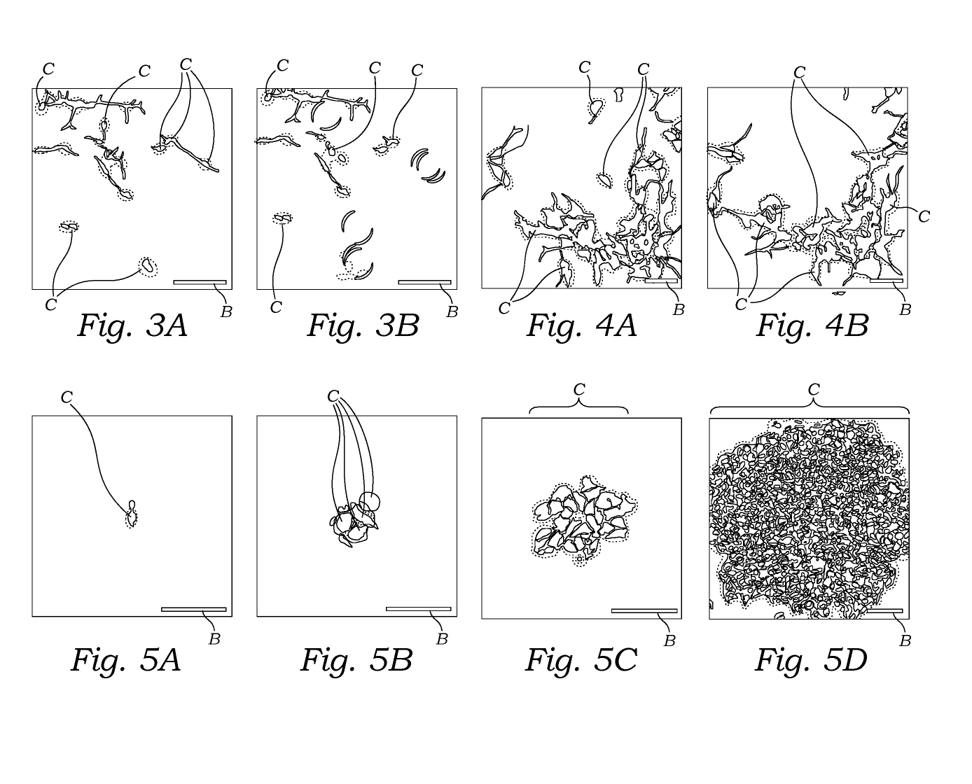

Turning now to FIG. 2, there is shown a perspective view in reduced scale of the exemplary sampling apparatus 10 of FIGS. 1A and 1B operably engaged with an exemplary linear actuator assembly A, again such as the live cell and tissue acquisition system (“CTAS”) as disclosed in WO/2008/021202 (“CTAS-Live”). Such a CTAS-Live system A is shown as generally having a central filter or filter coupling F, which connects to a valve (not shown) that supplies the vacuum, and with a substantially concentric adjustable ring light R thereabout, where the sampling apparatus 10 is then installed substantially axially via the luer hub 60 relative to the filter F. It will be appreciated that the head of the linear actuator assembly A is pivotable relative to the X-Y control stage S on which it is installed as to then shift the head and thus the sampling apparatus 10 to a substantially vertical orientation for access to a horizontally-oriented cell culture dish or the like (not shown). Micro adjustments by or to the X-Y control stage S then allow movement of the capillary tube 40, and particularly the “fine” first capillary tube section 42 (FIG. 1A), so as to acquire particular cells by substantially instantaneous vacuum, which will be further appreciated with reference to FIGS. 3-5 as discussed below. In operation, for example, with a sampling apparatus 10 as described and shown, such as having the first capillary tube inside diameter 44 of a nominal 15 μm, a vacuum strength of approximately 3.5-6.5 inches Hg and a vacuum duration of approximately 200 ms, individual live cells C (FIG. 1A0 can be collected within the collection tube 20 as by being effectively “sucked” through the capillary tube 40. Again, those cells C may remain viable through their deposit, as by gravity, in the growth medium 30. Those skilled in the art will appreciate that different vacuum strengths or durations, collection tube size or volume, and even capillary tube size may be appropriate in particular contexts, such that once again the exemplary specifications indicated are to be understood as merely illustrative of features and aspects of the present invention and non-limiting. A calibration LED source L for projecting the light on the tip of the capillary 40 is also shown.

Finally, turning now to FIGS. 3-5, there are shown schematic views of what are effectively microscope slide images of exemplary cells C and cell cultures as employed in connection with the present sampling apparatus 10 (FIGS. 1-2). It is noted that with all such “slides,” the scale bar B at the lower right of each figure refers to a true scale dimension of 100 μm, though again, due to a number of factors none of figures are to be taken as literally being “to scale,” the scale bar B here simply assisting with proportionality. For example, in FIG. 3A there is shown primary rat neuroprogenitor cultures before collection, with exemplary cells C identified. Then, in FIG. 3B the same primary rat neuroprogenitor cultures are shown after collection of the cells C, which evidences the ability of the system and apparatus to substantially precisely collect live single cells in situ from native tissues or cell cultures without cell death (see particularly FIG. 5) and without otherwise adversely affecting the cells, cell culture, or any native heterogeneous tissue. Similarly, in FIGS. 4A and 4B there are shown SH-SY5Y human neuroblastoma cells before and after collection, respectively. Again, it can be seen through examining the “before” and “after” slides that where some of the cells were previously they are no more after collection, now having been pulled into the sampling apparatus 10 as above-described, while still showing no real adverse effect on the remaining cells C or the overall culture. Regarding cell viability even after collection, shown in FIGS. 5A-5D are schematic microscope slides representative of re-cultivation of a single CHO cell, showing that the collected cell(s) C are viable. That is, the sequential panels or slides from FIG. 5A to 5D demonstrate representative clonal expansion of a single CHO cell C collected with the DCU-Live sampling apparatus 10 using the CTAS-Live linear actuator assembly A. The latter indicates single cell resolution capabilities of the described DCU-Live and viability of the collected cells C.

To summarize, regarding the exemplary embodiments of the present invention as shown and described herein, it will be appreciated that a sampling apparatus is disclosed and configured for collection and preservation of live cells from tissues and cell cultures. Because the principles of the invention may be practiced in a number of configurations beyond those shown and described, it is to be understood that the invention is not in any way limited by the exemplary embodiments, but is generally directed to a disposable capillary unit (DCU-Live) sampling apparatus configured for operable engagement with a live cell and tissue acquisition system (“CTAS”) such as disclosed in WO/2008/021202 (CTAS-Live) and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Furthermore, while aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear here, that the inventor believes that the claimed subject matter is the invention.

What is claimed is:

1. A sampling apparatus for collection and preservation of live cells, comprising:
a collection tube;
a capillary tube extending distally and substantially axially from the collection tube, the capillary tube having a first capillary tube section defining a first capillary tube inside diameter; and
a luer hub engaged proximally and substantially axially on the collection tube substantially opposite the capillary tube, the luer hub having a luer passage formed therethrough, whereby installation of the sampling apparatus in a linear actuator assembly capable of providing a vacuum as by engaging the luer hub with a filter coupling of the linear actuator assembly enables selective suctioning through the luer passage, the collection tube, and the capillary tube so as to collect one or more cells within the collection tube.

2. The apparatus of claim 1 wherein the collection tube is formed having an internal collection tube buffer configured to be substantially coaxial and contiguous with a second capillary tube section of the capillary tube.

3. The apparatus of claim 2 wherein the collection tube buffer and the second capillary tube section are unitary.

4. The apparatus of claim 2 wherein the collection tube buffer and the collection tube are unitary.

5. The apparatus of claim 2 wherein a growth medium is deposited within the collection tube substantially about the collection tube buffer.

6. The apparatus of claim 1 wherein the collection tube is formed having a collection tube body having collection tube markings thereon.

7. The apparatus of claim 1 wherein the capillary tube is further formed having a second capillary tube section of the capillary tube defining a second capillary tube inside diameter that is larger than the first capillary tube inside diameter.

8. The apparatus of claim 1 further comprising a folding cap installed substantially between the collection tube and the luer hub.

9. The apparatus of claim 8 wherein the folding cap is formed having a first folding cap member configured to engage a first luer hub boss of the luer hub and having a second folding cap member selectively engageable with the first folding cap member and configured to engage the collection tube.

10. The apparatus of claim 9 further comprising a folding cap hinge interconnecting the first and second folding cap members.

11. The apparatus of claim 1 wherein the luer hub is formed having a first luer hub boss and a second luer hub boss projecting proximally and substantially axially therefrom.

12. The apparatus of claim 11 wherein the second luer hub boss is formed having one or more luer hub engagement tabs.

13. A sampling system for collection and preservation of live cells, comprising:
a sampling apparatus comprising:
a collection tube having a growth medium therein;
a capillary tube extending distally and substantially axially from the collection tube, the capillary tube having a first capillary tube section defining a first capillary tube inside diameter; and
a luer hub engaged proximally and substantially axially on the collection tube substantially opposite the capillary tube, the luer hub having a luer passage formed therethrough; and
a linear actuator assembly capable of providing a vacuum, comprising:
a filter coupling configured for removable receipt of the luer hub so as to be in fluid communication with the luer passage and thus to operably accept the sampling apparatus; and
an X-Y control stage operably connected to the filter and configured for selective positioning of the sampling apparatus installed therein;
whereby installation of the sampling apparatus in the linear actuator assembly as by engaging the luer hub with the filter coupling of the linear actuator assembly enables selective suctioning through the luer passage, the collection tube, the collection tube buffer, and the capillary tube so as to collect one or more cells within the growth medium of the collection tube.

14. The system of claim 13 wherein:
the collection tube is formed having an internal and substantially coaxial collection tube buffer; and
the growth medium is deposited within the collection tube substantially about the collection tube buffer.

15. The system of claim 14 wherein the capillary tube is further formed having a second capillary tube section proximal of and contiguous with the first capillary tube section and distal of and substantially coaxial and contiguous with the collection tube buffer.

16. The system of claim 13 further comprising a folding cap installed substantially between the collection tube and the luer hub.

17. A sampling apparatus for collection and preservation of live cells, comprising:
a collection tube formed having an internal and substantially coaxial collection tube buffer and a growth medium deposited within the collection tube substantially about the collection tube buffer;
a capillary tube extending distally and substantially axially from the collection tube, the capillary tube having a first capillary tube section and a second capillary tube section proximal of and contiguous with the first capillary tube section and distal of and substantially coaxial and contiguous with the collection tube buffer; and
a luer hub engaged proximally and substantially axially on the collection tube substantially opposite the capillary tube, the luer hub having a first luer hub boss and a second luer hub boss projecting proximally and substantially axially therefrom and further having a luer passage formed therethrough so as to communicate between the first and second luer hob bosses, whereby installation of the sampling apparatus in a linear actuator assembly capable of providing a vacuum as by engaging the luer hub with a filter coupling of the linear actuator assembly enables selective suctioning through the luer passage, the collection tube, the collection tube buffer, and the capillary tube so as to collect one or more cells within the growth medium of the collection tube.
